(19) United States
(12) Patent Application Publication
(10) Pub. No.: US 2012/0225445 A1
(43) Pub. Date: Sep. 6, 2012

(54) SEBOCYTES, SEBOCYTE CELL LINES AND APPLICATIONS THEREOF

(76) Inventor: Christos Zouboulis, Berlin (DE)

(21) Appl. No.: 13/248,370

(22) Filed: Sep. 29, 2011

Related U.S. Application Data
(63) Continuation of application No. 12/243,869, filed on Oct. 1, 2008, now abandoned.
Application No. 12/243,869 is a continuation of application No. 09/920,392, filed on Aug. 1, 2001, now abandoned.

(30) Foreign Application Priority Data
Feb. 1, 1999 (DE) 19903920.8

(51) Int. Cl. C12N 5/077; C12Q 1/02; C12P 33/00; C12N 15/64; C12P 21/00; C12P 7/64; C12N 5/10; C12P 1/00

(52) U.S. Cl. 435/29; 435/366; 435/41; 435/91.4; 435/70.3; 435/134; 435/52

(57) ABSTRACT

Adipose cells (sebocytes) are described, The invention especially relates to sebaceous gland cells and to a sebaceous gland cell line with the property of being continuously grown over many sub-cultures. The sebocytes are excellently suited for useful applications.

### STATEMENT OF RELATED APPLICATIONS

This application is a continuation of U.S. patent application Ser. No. 12/243,869, filed Oct. 1, 2008, entitled “Sebocytes, Sebocyte-Cell Line and Uses Thereof,” which is a continuation of U.S. patent application Ser. No. 09/920,392, filed Aug. 1, 2001, entitled “Sebocytes, Sebocyte-Cell Line and Uses Thereof,” both of which are incorporated herein by reference in their entireties.

### DESCRIPTION

The present invention relates to grease or lipid containing and sebum producing cells of the skin and the mucous membrane, also called sebocytes. The present invention particularly relates to cells of the sebaceous gland and a cell line of sebaceous gland with the property of being capable to be continuously cultured through a large number of sub-cultures. The sebocytes are particularly suitable for useful applications, for example for the study of the physiology and the pathophysiology of human or animal sebaceous glands, for the study of the generation of acne, seborrhoe or other diseases, for testing the effectiveness of various substances and of medicaments, for the development of cell culture systems being based on two-dimensional or three-dimensional cell assemblies and constructions of organ-like structures, and for the manufacture of products being derived from these cells.

### BACKGROUND ART

Increasing indications: suggest that sebocytes play a critical role in the pathophysiogic processes and diseases of the sebaceous gland/hair complex, in particular in acne (Gollnick et al. J. Dermatol. 1991; 18:489-499; Brown and Shalita, Lancet 1998; 351:1871-1876; Cunliffe, Dermatology 1998, 196:9-15; Strauss, Dermatology 1998; 126:182-184) The majority of our understanding of the physiology and pathophysiology of the sebaceous gland derive from experimental animal models (Pochi in “Models in Dermatology”, Vol 2, N. Lowe and H Maibach, editors, Basel, 1985; 70-75). However, it was found that animal models do not allow reasonable predictions for the evaluation of the effectiveness of anti-acne medicaments for humans (Geiger, Dermatology 1995; 1991:305-310). The fact that acne occurs only in humans and that the secretion activity of the sebaceous gland is strongly species specific (Nikkari, J. Invest. Dermatol. 1974; 257-267) led to the search for human models. Preliminary studies for the avoidance of these disadvantages was carried out with human skin samples, which had been either incubated in-vitro (Hsia et al., Proc. Soc. Exp. Biol. Med. 1970; 135:285-291; Cooper et al., Br. J. Dermatol. 1976; 94:156-172; Sharp et al., J. Endrocrinol. 1976; 70:491-499), or had been transplanted to nude mice (Petersen et al., J. Clin. Invest. 1984; 74:1358-1365). Basic studies on the activity of human sebocytes and their regulation were made possible only in the recent decade, as vital human sebaceous glands were isolated (Kealex et al. Br. J. Dermatol. 1986; 114:181-188) and a culture model for human sebocytes could be established in vitro (Xia et al., J. Invest, Dermatol. 1989; 93:315-321).

By means of modifications of the culture technique of Xia et al. (1989), improvements have been achieved during the recent years in view of reproducibility of the cultivation of human sebocytes in vitro. Thus, Zouboulis et al. (Skin. Pharmacol. 1991; 4:74-83) omitted hydrocortisol in the culture medium by means of adding human serum. Lee (in Epithelia: Advances in Cell Physiology and Cell Culture; C. J. Jones, editors: Kluwer, Dordrecht, 1990; 333-350) treated sebaceous glands with collagenase before cultivating them in serum-free medium which was enriched with additives. Also, primary sebocyte cultures were obtained by omitting the 3T3 fibroblast cell layer which served as an adherence base layer (Akamatsu et al., J. Invest. Dermatol. 1992; 99: 509-511). Secondary cultures were kept in a medium which was supplemented by lipid free serum (Zouboulis et al., J. Invest. Dermatol. 1993; 101:628-633), and in serum-free ceratinocyte containing basal medium without additives (Akamazu et al., J. invest. Dermatol. 1992; 99:509-511). In addition, it was shown that the keratinocyte growth factor (KGF) remarkably increases the yield and the proliferation of human sebocytes (Chen et al., J. Invest. Dermatol. 1998; 110:84-89).

In spite of these technical improvements, further progress is strongly hampered by the situation that a cultivation of a large number of sebocytes from isolated human sebaceous glands is difficult. In particular, there is the difficulty to keep the cell material in culture for a long period of time. As the reason therefore, it is assumed that the sebocytes tend to differentiate and to die via spontaneous cell membrane rupture and the subsequent release of their content. The best result yet achieved was that of Fujie et al. (Arch. Dermatol. Res. 1996; 288:703-708), who isolated sebaceous glands on the basis of the technique of Xia et al. (1989) and cultivated sebocytes by means of the method of dispersed cell culture through six sub cultures in serum-free, keratinocyte growth medium without a cell adherence layer.

### SUMMARY OF THE INVENTION

It is an object of the present invention to provide sebocytes (sebaceous cells) which can be maintained in culture through higher number of subcultures. In this context, the provided sebocytes shall approach, in the appearance of their morphological, phenotypic and functional characteristics, those of viable, normal human sebocytes, at least to such an extent that they are suitable as cellular material or a cell culture model for lipid containing, sebum-producing cells, and in particular for sebocytes, for physiological, pathophysiological and pharmaceutical evaluations and studies.

The object is solved by the provision of sebocytes which are immortalised. Suitably, the cells of the present invention derive from humans, because this is of primary interest for useful applications. Sebocytes of this kind are present in the human sebaceous gland cell line SZ95, which have been deposited with the German collection of microorganisms and cell cultures (DSMZ) under the depository No. DSM ACC2383.

### DESCRIPTION OF THE DRAWINGS

FIGS. 1a-1c shows (a) normal, human sebocytes of the second subculture, from which the provided immortalised sebocytes derive, (b) an adherent sebocyte culture as provided by immortalised secocytes from the primary sub culture, and (c) immortalised sebocytes (50th subculture of a clone). All cells exhibit a similar ephithelial, polymorphous structure.

FIGS. 2a-b shows cyto-centrifuged samples of (a) immortalised sebocytes monoclonal antibody against SV40 large T antigene which resulted in a strong, mostly nucleic, partly cytoplasmatic staining, and (b) Normal keratinocytes and fibroplasts were uniformly negative vis-a-vis the SV-40 large T protein and the HMEC-1 cells as a positive control showed mostly a nucleic, partly cytoplasmatic staining for the SV-40 large T protein.

FIGS. 3a-b shows the immunocytochemical results on cytocentrifugation preparations of: (a) the immortalized sebocytes which exhibited a positive cytoplasmatic staining and (b) normal human epidermal keratinocytes which were not stained. The preparations were stained with a monoclonal antibody against the sebaceous gland antigene.

FIG. 4 shows the expression of the keratines 7, 13 and 19 as well as various proteins of the human polymorphous epithelial mucine group in the immortalized sebocytes. Expression was detected by Western blot analysis, whereas human keratinocytes expressed only keratine 13.

FIG. 5 shows the staining with Nile Red and the assessment by fluorescence microscopy showed the presence of lipids in the cell cytoplasm resulted in individual or in grouped lipid droplets which were optionally divided in the cytoplasm of the sebocytes.

FIG. 6 shows HPTLC-fractionized lipids were detected after pulse recording of radioactively labeled sodium acetate by means of radiometric image evaluation with two selected immortalized and cloned sebocyte cultures (see lanes 3 and 4 as well as 5 and 6, respectively).

FIG. 7 shows the proliferation of an immortalized cloned sebocyte cell line (SZ95) over 18 days in sebocyte medium.

FIG. 8 shows an exemplary sebocyte clone where the proliferation of the cells (seeding of 2.000/well) was observed over 18 days in serum-free medium (control) as well as in serum-free medium which was supplemented with 10⁻⁶ M 5α-DHT.

FIGS. 9a-9d shows inhibition of proliferation by retinoids (typically distinctively pronounced in the order of 13-cis-retinoic acid>all-trans-retinoic acid>>Acitretin) of a) immortalized Sebocytes/Clone-6, b) immortalized Sebocytes/clone-7, and c) immortalized Sebocytes/Clone-28; clones were stimulated in proliferation (for example by all-trans-retinoic acid and 13-cis-retinoic acid) corresponding to the proliferation response of normal human epidermal keratincoytes (FIG. 9d).

### DESCRIPTION OF THE PREFERRED EMBODIMENTS

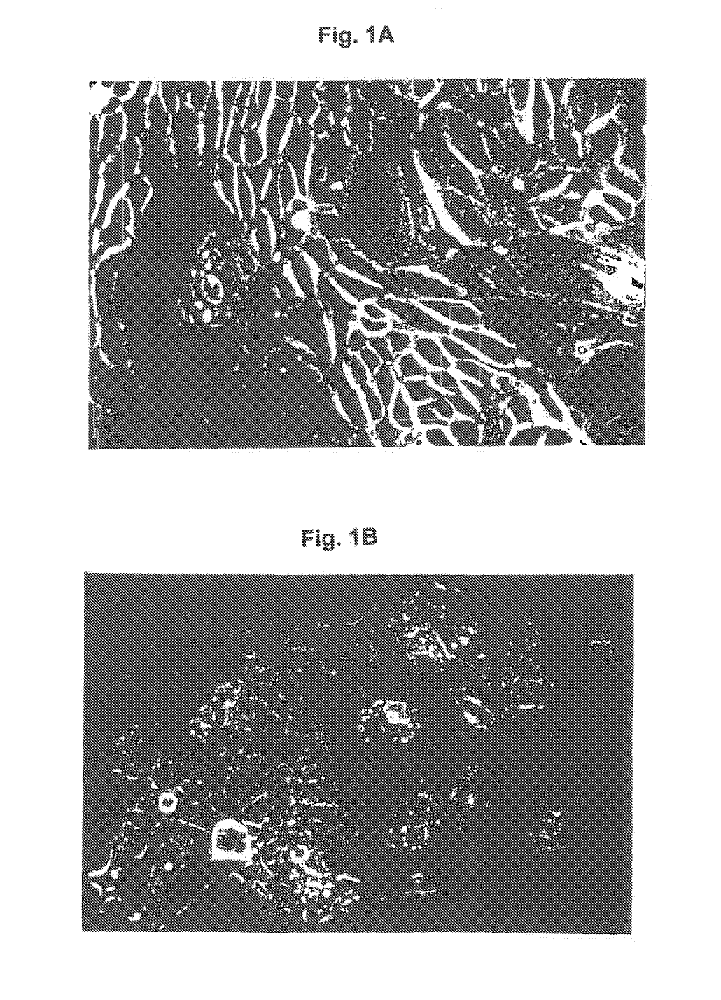

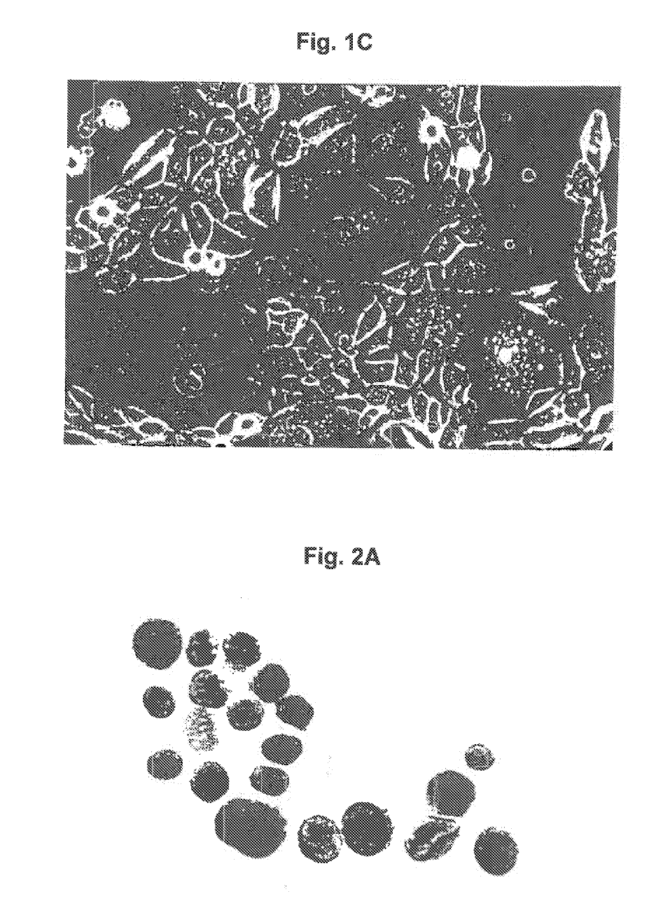

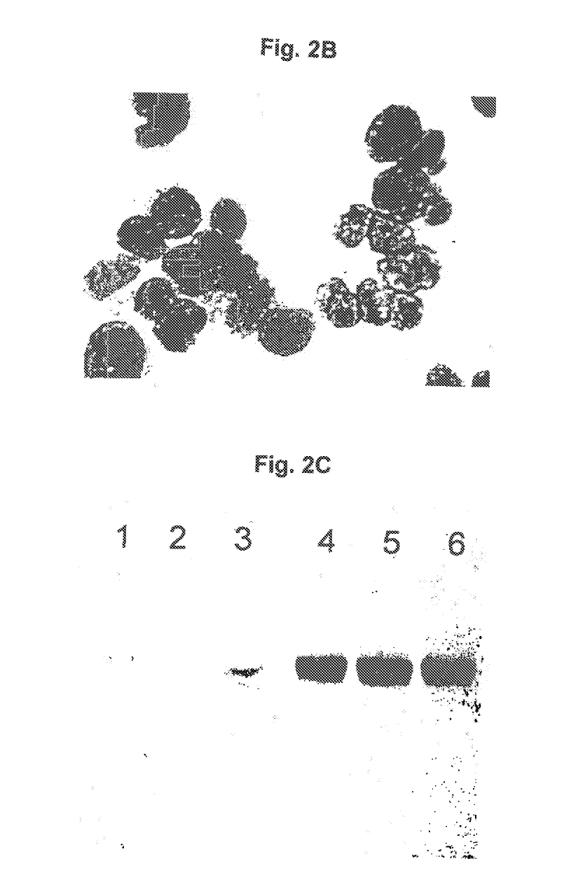

The present invention will, in the following, be described in more detail with reference to the drawings (Figs.). FIG. 1 and FIG. 2 show that immortalised sebocytes SZ95 provided by the invention have maintained the epithelial, polymorphous appearance of primary, normal sebocytes, from which they derive (in the present case: from humans). In addition, the provided immortalised sebocytes and the clones thereof (a clone means cells which surely derive from a single cell) express the characteristic 94-kD-large SV-40 large T-antigen, with which coding DNA sequence transfection had been carried out, even in the later subcultures. FIG. 1 shows (a) normal, human sebocytes of the second subculture, from which the provided immortalised sebocytes derive, (b) an adherent sebocyte culture as provided by immortalised secocytes from the primary sub culture, and (c) provided immortalised sebocytes (50^{th} subculture of a clone) All cells exhibit a similar ephithelial, polymorphous structure. FIG. 2 shows cyto-centrifuged samples of (a) provided immortalised sebocytes, and (b) of endothelial cell culture cells HMEC-1 which served as a positive control, both having been labeled with a monoclonal antibody against human SV-40 large T-antigen. Both samples are labeled positively and show that the human SV-40 large T-antigen is present predominantly in the cell nucleus, partly also in the cytoplasm of the cell. In (c), the expression of human SV-40 large T-antigen in the provided immortalised sebocytes is demonstrated by means of Western blot analysis. While human SV-40 large T-antigen was not detectable in the non-transfected, normal human sebocytes (lane 1) and—in normal human epithelial keratinocytes (lane 2), the characteristic 94-kD large protein was determined in protein extracts of the provided immortalised sebocytes in the 34.sup.th subculture (lane 3) as well as in three isolated clones (lanes 4, 5 and 6).

The immortalised sebocytes of the present invention are preferably of human origin. The meaning of the expression “sebocytes” is to be understood in the broadest sense, i.e. relating to all cells which are, more or less, grease- or lipid-containing and sebum-producing. Sebum is merely composed of various fatty or lipid substances. In this connection, the fat or lipid content of the cells may vary in terms of the lipid substance fraction as well as in terms of content of the lipid substance fractions. As a rule, but not necessarily, the fatty or lipid substance content of the cells comprise free fatty acids, triglycerides, wax, squalene, free cholesterol, cholesterol esters, dihydroxy cholesterol and other steroids as well as hydrocarbons. In particular, those immortalised sebocytes are preferred which derive from human sebaceous gland cells. A particularly good suitability for medicinal purposes is achieved, if the sebocytes derive from human sebaceous gland cells of the face.

An essential characteristic of the sebocytes of the invention is their immortalization Immortalised within the meaning of the present invention basically means maintaining the vital condition of the cells through multiple subcultures. The immortalised sebocytes SZ95 of the present invention could be maintained in culture, in the past observation time of about 4½ years, over more than 50 subcultures, whereas normal human sebocytes can only grow up to three to six subcultures before they die.

The immortalised sebocytes according to the present invention can be obtained by means of transfecting normal sebocytes-using, in the preferred embodiment, those of human origin and particularly of the human sebaceous glands—with a DNA which act on forming stable, inactive complexes with proliferation inhibiting genes. A particularly successful immortalisation was achieved in the present invention by transfecting normal human sebocytes, in particular those derived from sebaceous glands of the face, with a DNA comprising DNA sequences which encode for the large T-protein of SV-40. The immortalising effect of the SV-40 large T-antigen (protein) as well as the corresponding use of the coding DNA sequence for transfecting human cells is basically known. Thus, immortalised cell lines had been obtained through transfection with a DNA encoding for SV-40 T, for example with other cells of epithelial origin (see Tohyama et al., Tohoka. J. Exp. Med. 1997; 182:75-82; Bae et al Prostate 1998; 34:275-282), as well as of endothelial origin (see Ades et al., J. Invest. Dermatol. 1992; 99:683-690 and WO-A-92/17569).

It was found that by transfecting sebocytes with a gene transfer method by means of applying cationic lipids (LTPOTECTIN reagent, which is an 1:1 (w/w) liposomal formulation containing the cationic lipids DOTMA [1,2-Diolyloxy propyl-3-trimethyl ammonium chlorid] and DOPE [Diolylphosphatidylentanolamin] in membrane filtered water [1 mg/ml]), in which foreign DNA is taken up through endocytosis into the cells via cationic lipid/DNA complexes (see Wang et al., In. Vitro. Cell. Dev. Diol. 1991; 27A:63-74; Staedel et ale, J. Invest. Dermatol. 1994; 102:768-772), good results were achieved for the immortalisation, while the transfection mixture preferably contained in addition 0.25 to 2.0 vol.-% and particularly 2.0 vol.-% LIPOFECTIN reagent as well as 0.05 to 0.5 wt.-% and particularly 0.5 wt.-% foreign DNA in a suitable transfection buffer The foreign DNA, like the one coding for the SV-40 large T-protein, is typically inserted in a suitable vector, by which the expression of the SV-40 large-T-protein is enhanced, preferably by means of promotor and enhancer sequences. If normal human sebocytes are transfected, in the preferred embodiment, by the DNA which encodes for the SV-40 large T-antigen, it is to be expected that the provided sebocytes express the large T-antigen of SV-40 after a successful transfection and immortalisation. This was confirmed for the immortalised sebocytes provided according to the present invention by immuno-cytochemical means and by means of Western blot analysis, using monoclonal antibodies against the SV-40 large T-antigen.

The thus obtained, immortalised sebocytes are preferably in the state of a cell line which, in this form, can be excellently used for the application purposes.

The immortalised sebocytes according to the present invention did grow, after their adaptation to serum-free culture medium, better than non-transfected, normal human sebocytes, and they maintained the capability of synthesising sebaceous gland specific lipids—contrary to the non-transfected normal human sebocytes which had been maintained in serum-free medium The immortalised sebocytes according to the present invention can thus serve as a continuously renewable and propagating cell line and can grow in defined culture media.

A particular value of the irmortalised sebocytes according to the present invention is that they have features of non-transfected, normal and differentiated sebocytes in morphological, phenotypic and functional respects Therefore, the immortalized sebocytes according to the present invention can be excellently offered as models for physiological, pathophysiological and pharmacological studies At the same time, the disadvantage of limited viability of conventional, cultured normal sebocytes of human origin is avoided. Accordingly, it was confirmed that the immortalised sebocytes according to the present invention can substantially maintain the phenotype of normal sebocytes and can behave like non-transfected normal human sebocytes of the face in functional respects.

It was found that the immortalised sebocytes or the sebocyte cell line according to the present invention exhibit a polymorphous, epithelial appearance which is similar to that of non-transfected, normal human sebocytes. In cell culture, cells did grow in various sizes and intracellular structures, which is indicative for various phases in cell maturation. Thus, cells of various sizes, in the average up to the 5-fold or 6-fold size with confluent growth, had been observed, which essentially corresponds to the cell growth increase of non-transfected, normal human sebocytes with progressive differentiation in vitro (in the average 4-fold to 5.5-fold size difference). Furthermore, the immortalised sebocytes according to the present invention was found to be rich in fatty substance or lipid particles in the cytoplasm, like in non-transfected, normal human sebocytes. The synthesis of the characteristic sebaceous gland lipids squalene and wax esters, which are common for normal human sebocytes, was confirmed experimentally in the course of the present invention. Furthermore, the immortalized sebocytes of the present invention synthesised free fatty acids, which, again, correlates with the findings of non-transfected, normal human sebocytes in vitro—and even after a high number of subcultures.

Also, expression markers which confirm a sebocyte origin and which indicate a viable differentiation, were confirmed as typical indications for sebocytes of the immortalised sebocytes or sebocyte cell line of the present invention. Thus, the immortalised sebocytes or the sebocyte cell line expressed antigens which are typical for the human polymorphous epithelial mucous protein group, such as the sebaceous gland antigen, the human milk fat globulins 1 and 2, the human epithelial sialomucine, the Thomsen-Friedenreich antigen, the mucin-like carcinogen associated antigen and the epithelial membrane antigen. This was confirmed in the course of the present invention by immuno cytochemical means and by means of Western blot analysis. In addition, the immortalised sebocytes or the sebocyte cell line of the present invention expressed keratinic antigens typical for non-transfected, normal human sebocytes, such as those of subclasses 7, 13 and 19. The antigen phenotype thus demonstrated the sebocyte origin as well as the differentiation of the sebocytes.

Also in functional terms, the immortalised sebocytes or the sebocyte cell line of the present invention are similar to non-transfected, normal human sebocytes. Thus, the immortalised sebocytes of the present invention responded to the effects of androgens, such as e.g. by 5α-dihydro testosterone, by enhancing their in vitro proliferation. In addition, the immortalised sebocytes or the sebocyte cell line of the invention possessed the capability of varying their proliferation by the effects of retinoids, in particular those of the non-aromatic type (e.g. 13-cis-retinoic acid, all-trans-retinoic acid).

A preferred embodiment of the present invention is that the immortalised and preferably the human sebocytes are cloned. This is advantageous because the immortalised sebocytes or the thus generated sebocyte cell line are well defined and specifically characterized by means of their unique genomic basis. A cloned and immortalised human sebocyte cell line was suitably obtained by gradually diluting immortalised sebocytes in culture vessels long enough, until the cell division started again from only one cell per culture vessel. This could be observed and controlled by means of microscopic observations.

Accordingly, it was found in the course of the present invention that the obtained immortalised human sebocytes or the thus obtained sebocyte cell line essentially maintained the sebocyte identity compared to non-transfected, normal human sebocytes. This was confirmed by characteristic determinations and functional tests.

A sebocyte cell line with the specification SZ95, which entails the above-mentioned advantages of the present invention, is represented by the sebaceous gland cell line which was deposited with the DSMZ under the depository No. ACC2383.

Accordingly, the immortalised sebocytes or the sebocyte cell line according to the present invention offer excellent possibilities for useful applications. In general, the sebocytes or the sebocyte cell line according to the invention can be used for diagnostic, for therapeutic, or for cosmetic: uses. Specifically, the sebocytes or the sebocyte cell line described above may serve for developments and studies of the physiology or the pathophysiology of lipid-containing, sebum producing cells, in particular of human or animal sebaceous gland cells, as well as their role in pathophysiologic processes of the skin and in skin diseases like, e.g, acne. With the help of the invention the generation of acne and/or seborrhoe and/or other diseases, especially of skin diseases in which the sebaceous gland function plays a role or may play a roll, can be studied The products of the present invention further serve as excellent models for testing and for evaluating anti-acne compounds and/or anti-seborrhoe compounds or therapeutic agents, but also therapeutic agents against diseases, especially skin diseases, in which the sebaceous gland function plays a role or may play a role. Especially for the performance of clinical studies, such in vitro studies on pharmacological properties of medicaments are useful.

The sebocytes or the sebocyte cell line of the invention as described above additionally have the advantage that further cell culture systems may be established. This includes the development of simple and of complex cell culture systems. Simple cell culture systems means, as a rule, two-dimensional one-layer or multi-layer adherent cultures or non-adherent cultures and are, for example, formed by means of addition of the above-described sebocytes to other cell types, or by means of cultivating them through semi- or non-permeable membranes (Schwartz et al., J. Surg. Res. 1998; 76: 79-85; Nackman et al. Surgery. 1998; 124: 353-361). Complex cell culture system means, as a rule, a three-dimensional cultivation of one-layer or multiple-layer cultures and are, for example, formed by Cultivating the cells as spheroids, on spheroids, in collagen or in other jelly materials or in an artificial skin-like structure (Korff and Augustin, J. Cell. Biol 1998; 143:1341-1352; Hamamoto et al.; J. Biochem., (Tokyo) 1998; 24:972-979; Desoize et al. Anticancer. Res. 1998; 18.4147-4158; Hamilton, Cancer. Lett. 1990; 131:29-34; Niemann et al., J. Cell. Biol. 1998; 143:533-545; Awata et al., J. Gastroenterol. Hepatol. 1998; 13Suppl:S55-61; Voura et al.r Microsc. Res. Tech. 1998; 43:265-275; Pipili-Synetos et al., Br. J. Pharmacol. 1998; 125:1252-1257; Vasile et al., J. Histochem. Cytochem. 1999; 47:159-168; Michalopoulos et al. Hepatology. 1999; 29:90-100; Trent and Kirsner., Int. J. Clin. Pract. 1998; 52:408-413; Fransson et al., Br. J. Dermatol. 1998; 139:596-604; Konstantinova et al., Arch. Dermatol. Res. 1998; 290:610-614; Black et al., FASEB J. 1998; 12.1331-1340; Zhao et al., Biochem. Biophys. Res. Commun. 1999; 254:49-53).

A particularly useful application relates to the generation of three-dimensional cell assemblies, or constructions or reconstructions of organ-like structures based on the sebocytes or the sebocyte cell line of the invention. For this purpose, the sebocytes are used alone, but preferably in addition to further skin generating cells, in particular with keratinocytes, fibroblasts, melanocytes, endothelial cells, Langerhans' cells and/or cells from the hair follicle. For the generation of three-dimensional cell assemblies, or constructions or reconstructions of organ-like structures, a support scaffold with collagen or other jelly materials and/or with parts of inactivated tissue is provided first, and then the aforementioned cells are applied in or onto this support scaffold This method is basically known to the man skilled in the art, and samples are commercially available (Trent and Kirsner, Int. J. Clin. Pract. 1998; 52:408-413; Fransson et al., Br. T. Dermatol. 1998; 139:589-604; Konstantinova et al., Arch. Dermatol. Res. 1998; 290:610-614; Black et al., FASEB. J. 1998; 12:1331-1340; Zhao et al., Biochem. Biophys. Res. Commun. 1999; 254:49-53). An “artificial skin” or a skin substitute is produced thereby, which offers excellent possibilities for the transplant or grafting medicine, for the reconstruction of damaged skin portions such as, e.g., burnt skin, or for the therapy of skin lesions. With the help of the present invention, such an “artificial skin” can synthesise lipids/sebum in sufficient amounts, when the sebocytes of the present invention are incorporated into the constructions.

A further field of useful applications relates to the manufacture of products which derive from the sebocytes or the sebocyte cell line of the invention This includes—the isolation and purification of cellular substances, such as lipids, proteins, DNA and/or RNA. Since the cells are immortalised, they are maintained to be offered as a continuous source for such cellular substances. Specific examples for very suitable substances, which can be obtained accordingly from these cells, include: skin lipids for their use in topical agents and medicaments, the antigenic proteins which are mentioned in Example 3 below in connection with a phenotypics characterisation of sebocytes, and, further, the generation of plasmid DNA or vector DNA. The generation of plasmid DNA or vector DNA is carried out by means of genetic engineering known to those skilled in the art. Accordingly, genes can be retrieved which induce lipid production. Especially with such suitable plasmid and vector constructions, which also includes the generation of viral vectors, again other cells or organisms can be modified or transfected.

The present invention will be explained in more detailed by reference to the following, non-limiting examples.

### EXAMPLES

For the examples described below, the following embodiments as to the materials and methods may be used. The examples should not be interpreted as being limiting.

### Cell Cultures

If not indicated otherwise, all cells were maintained in a medium as adherent cultures which consisted of a modified DMEM/HAM's F12-Medium (1:1) (available from Biochrom, Berlin, Germany) having 2 mM N-Acetyl-L-alanyl-L-glutamine which was supplemented with 10% heat-inactivated, fetal calf serium (FCS; Biochrom) as well as 50 μm/ml gentamycin (available from Gibco-BRL, Karlsruhe, Germany). The culture was maintained in a humid atmosphere containing 5% CO₂ at 37° C. and the culture medium was renewed every 2 to 3 days.

### Isolation and Cultivation of Normal Human Sebocytes

Normal sebocytes were isolated from the facial skin of a 87-year old female patient undergoing surgery, as reported by Xia et al. (J. Invest Dermatol. 1989; 93:315-321). The isolated sebaceous glands were cultured without feeder layer in the standard medium supplemented with 9 ng/ml epidermal growth factor (EGF), 9 ng/ml keratinocyte growth factor (KGF) (both available from Boehringer Mannheim, Germany), 0.4 μg/ml hydrocortisone (available from Sigma, Deisenhofen, Germany) as well as 10⁻⁹ M cholera toxin (available from Calbiochem, Bad Soden, Germany). Primary normal sebocyte cultures resulted as outgrowths from the periphery of the sebaceous gland lobules.

### Immunocytochemical Tests

Dispersed cells of sub-confluent normal sebocyte cultures were attached to glass slides by cytocentrifugation. The samples were air dried and fixed with cold acetone for 10 minutes. The preparations were subsequently incubated with the respective monoclonal antibody or a control antibody at room temperature for 30 minutes Bound antibodies were detected by coupling with a 1:100 dilution of a monoclonal antibody conjugate from rabbit/anti-mouse Ige (H+ L) and an alkaline phosphatase/anti-alkaline phosphatase complex (available from Dianova, Hamburg, Germany) at room temperature for 30 minutes. Primary and secondary monoclonal antibodies were diluted in solutions containing 10% RPMI-1640 and 10% FCS at a pH of 7.4. The washing steps were conducted three times with PBS buffer without Ca²⁺ and Mg²⁺ (available from Biochrom). The preparations were stained for 30 minutes in buffered solution (pH 8.8) with Neufuchsin as an adherent agent and a naphthol salt as a coupling agent (both from Sigma), counter-stained with Mayer's Haemalum (Merck, Darmstadt, Germany), covered and judged with a light microscopy.

### Isolation and Quantitation of Proteins

Cell cultures were washed twice with PBS, lysed directly in the culture dishes by a cold solution which consisted of 50 mM HEPES, 1% Nonidet P-40 (available from ICN, Aurora, Ohio, USA), 150 mM NaCl as well as a protease inhibitor (Complete™ Mini; available from Boehringer Mannheim) subsequently scrubbed and harvested in small centrifugation dishes to isolate cellular proteins. The obtained material was homogenized by ultrasonic disruption, subjected to centrifugation and the supernatants were held on ice, Bicinchoninic acid (BCA-protein assay; available from Pierce, Rochford, Ill., USA) was added to visualize the total protein and the protein concentration was quantitated by measurement of absorption at 550 nm.

### Western Blot Analysis

Aliquots of the isolated proteins (20 μg) were heated to 95° C. for 15 minutes, One-dimensional SDS/PACE electrophoresis was conducted with each sample on 7.5% gels. Then, proteins were transferred to a transfer membrane (Immobilon-P of PVDF; available from Millipore, Eschborn, Germany) utilizing a standard blot system (available from Bio-Rad, Munchen, Germany). The blots were incubated with primary monoclonal antibody at room temperature for 60 minutes and subsequently with horse radish peroxidase-conjugated goat/anti-mouse monoclonal antibody and goat/anti-rabbit monoclonal antibody, respectively (available from Oncogene Science) in a dilution of 0.2 μg/ml at room temperature for 60 minutes. After thorough washing, the signals were visualized by a chemiluminescence method utilizing a standard assay (ECL, available from Amersham, Braunschweig) on X-ray sensitive films (XAR 5; available from Kodak, Rochester, N.Y., USA), whereby various illumination intervals were adjusted.

### Oil Red and Nile Red Stamina

Cells grown in chamber slides were incubated either with 0.6% Oil Red (Sigma) in 60% isopropanol for 15 to 120 minutes or with 1 μg/ml Nile Red dye (available from Kodak) for 15 minutes at room temperature, as reported by Xia et al. (1989) (supra). The cultures were then observed under a light microscope (after Oil Red stain) or a fluorescence microscope using a 450 to 500 nm bandpass excitation filer by light emission of >580 nm (after Nile Red stain).

### Flow Cytometry

Dispersed, non-labeled cells were determined for their cell size using a conventional sorter, while cells labeled with Nile Red dye (available from Kodak) were assessed for lipid content by flow cytometry on a fluorescence basis. 10.000 cells per sample were tested.

### Labeling and Extraction of Lipids

Cell cultures were maintained in culture medium for 2 days and then radioactively pulsed via the sodium salt of [2⁻¹⁴ C]acetic acid (45-60 mCi/mmol; available from DuPont-NEN, Boston, Mass., USA) with a concentration of 0.5 μCi/ml in RPMI-1640 medium supplemented with 2 mM L-glutamine, 10% heat-inactivated FCS and 100 IU/ml penicilline and 100 μg/ml streptomycine. Incubation was continued for further 24 hours. Lipids were isolated from cultured cells and from the supernatant culture medium and separated into neutral lipids, fatty acids and phospholipids (see Seifert et al. J. Invest. Dermatol. 1997; 108: 375).

The size separation into fractions and the visualization of neutral lipids and free fatty acids was obtained by high performance thin layer chromatography (HPTLC) conducted on 20×10 cm² silica gel-coated glass plates (available from Merck, Darmstadt, Germany). The plates were pretreated with n-hexane and dried for 24 hours. The samples were applied by an automatic lipid applicator (Linomat IV; Camag, Berlin, Germany). Chromatograms of the neutral lipids were developed in a n-hexane-diethylether solution (9:1) on 9 cm, dried and post-developed in a solution of chloroform/diethylether, ethylacetate (80:4:16) on 4-5 cm. For illumination, illumination sheets (TR 2040S, available from Fuji, Tokyo, Japan) were used which were scanned using an image analyser (“BAS 1000 Bio-Imaging Analyser”, Fuji). Lipid standards were used as comparative samples.

### Growth Behavior Tests

Cells were seeded in 96 well culture plates at densities of 0.5 to 4×10³ cell/well. Cell proliferation was assessed by the 4-methylumbelliferylheptanoatefluorescence assay and measured automatically (Zouboulis et al. Melanoma. Res. 1991; 1:91-95).

To this extent, on the day of evaluation, the culture medium was removed, the cells were washed twice with PBS and 100 μl of a 100 μg/ml solution of 4-methylumbelliferyl heptanoate (Sigma, Deisenhofen, Germany) in PBS were added to each well. The plates were then incubated at 37° C. for 30 minutes and released fluorescence was measured by a suitable fluorescence measuring device (Titertec-Fluoroscan II; Flow, Meckenheim, Germany). Fluorescence units were obtained at 355 nm excitation and 460 nm emission filters.

Treatment with 5α-dihydrotestosterone and Retinoids

5α-dihydrotestosterone (5α-DHT; Sigma) was dissolved in DMSO and subsequently in serum-free, phenol-free modified DMEM/Ham's F12-Medium (1:1) (Gibco-BRL) with 2 mM n-Acetyl-L-alanyl-L-glutamine which was supplemented with 5 ng/ml EGF, 50 μg/ml bovine pituary extract, 1 mg/ml fatty acid-free bovine serum albumine (Boehringer Mannheim) and 50 μg/ml gentamycine to obtain a final concentration of 10⁻⁶ M 5α-DHT and 0.1% DMSO. 0.1% DMSO alone served as a control. The cells (0.5 to 2×10³/well) were treated with 5α-DHT for 18 days.

For the treatment with retinoids, all-trans-retinoic acid, 13-cis-retinoic acid and acitretin were dissolved in DMSO and subsequently placed in serum-free modified DME-Medium/Ham's F12-Medium (1:1) with 2 nM N-Acetyl-L-alanyl-L-glutamine which was supplemented with 5 ng/ml EGE, 50 μg/ml bovine pituary extract, 1 mg/ml fatty acid-free bovine serum albumine and 50.μg/ml gentamycine to obtain a final concentration of 10⁻⁷ M retinoid and 0.1% DMSO. 0.1% DMSO alone served as a control. Retinoids were handled under dim amber light. The cells (0.5 to 1×10³/well) were treated with the retinoids for 9 days.

### Statistical Analysis

Growth studies were assessed in sextuplicate formulations of 96 well plates. All other experiments were performed in triplicate formulations.

### Example 1

### Transfection of Normal Human Sebocytes

The vector used for the transfection of normal human sebocyte designated pSVT was a plasmid construct on the basis of PBF322 comprising sequences for the SV-40 large T protein where its protein expression was driven by the Rous Sarcoma Virus long-terminal repeat (see Dutt et al. Oncogene 1990; 5:195-200; Wang et al. In. Vitro. Cell. Dev. Biol. 1991; 27A:63-74). Human sebocyte cultures in the second subculture were grown to 50% confluency in 35 mm culture dishes (Becton Dickinson, Plymouth, USA) and used for transfection. The transfection was performed on the basis of a gene transfer method using cationic lipids. To this extent, the LIPOFECTIN reagent was utilized which contained a 1:1 (w/w) liposomal formulation of cationic lipids DOTMA (1,2-Diolyloxypropyl-3-trimethylammo-niumchloride) and DOPE (Diolylphosphatidylethanolamine) in membrane filtered water (1 mg/ml). To this extent, the culture medium was removed, the culture cells were washed twice with serum-free medium (Opti-MEM from Gibco-BRL) and incubated in this medium for 4 hours. The medium was then replaced by a transfection mixture consisting of an antibiotic-free amount of Opti-MEM medium (1.5 ml) with a suitable amount of the LIPOFECTIN reagent (Gibco-BRL; 5-30 μl, most preferably 1.5 vol-%) as well as a suitable amount of pSVT DNA (1-10 μg) in a solution having 0.5 ml PBS (final DNA concentration, most preferably 0.5 wt.-%). The cultures were incubated in humid atmosphere containing 5% CO₂ at 37° C. for 24 hours. The cultures were finally washed twice with culture medium and further maintained in sebocyte culture medium as described above.

After the transfection treatment, a drastically diminished viability of the pSVT-treated sebocytes was observed during 4 weeks. Particularly with the use of optimal amounts of LIPOFECTIN reagent and pSVT DNA proliferating sebocyte colonies occurred. These cells (SZ95) were able to be passaged to date more than 50 times. They are still viable upon the observation period of 4.5 years.

### Example 2

### Cloning of Immortalized Human Sebocytes

The thus immortalized human sebocytes SZ95 were seeded in 96 well culture plates using a dilution series with geometrically descending cell numbers of 1×10² cells in the first series until theoretically zero cells were reached in the last series (Zouboulis et al. in “The malignous melanome of the skin”, C. E. Orfanos and C. Garbe (eds.) Zuckschwerdt, Munchen, Germany: 1990; 158-168). The cells were maintained in standard culture medium supplemented with 5 mg/ml ECF and 3 ng/ml KGF. Growing cells were regarded as clones in case they were derived from a single cell per well which was observable by light microscopic experiments. Thus, cloned SZ95 cells were obtained.

### Example 3

### Characterization of the Immortalized Human Sebocytes

### Detection of SV-40 Large T Antigene

The expression of SV-40 large T antigene in immortalized sebocytes was detected immunocytochemically and by Western blot analysis using a monoclonal anti-human SV-40 large T antigene antibody from mouse serum (Oncogene Science, Cambridge, Mass., USA) which was diluted for immunocytochemical analysis to 1:1000 and for Western blot analysis to 1:100. Human normal epidermal keratinocytes, dermal fibroplasts and as a positive control endothelial cells HMEC-1 immortalized by SV-40 large T antigene (see WO-A-992/175 69) were used as a comparison.

The immunocytochemical experiment of the immortalized sebocytes according to Example 1 with the monoclonal antibody against SV40 large T antigene resulted in a strong, mostly nucleic, partly cytoplasmatic staining (see FIG. 2a). Normal keratinocytes and fibroplasts were uniformly negative vis-a-vis the SV-40 large T protein and the HMEC-1 cells as a positive control showed mostly a nucleic, partly cytoplasmatic staining for the SV-40 large T protein (see FIG. 2b).

FIG. 2c shows the results of Western blot analysis of SV-40 large T antigene expression in non-transfected normal human sebocytes (lane 1), in normal human epidermal keratinocytes (lane 2), in immortalized sebocytes according to the present invention (34^{th} sub-culture; lane 3) as well as in various cloned sebocytes according to the present invention (lanes 4, 5 and 6). A band at 94 kD was confirmed to be the immortalized sebocyte line as well as its clones which confirmed the expression of the SV-40 large T protein (see Harlow et al. J. Virol. 1981; 39:861-869).

### Phenotypic Characterization of the Immortalized Sebocytes According to the Present Invention

The morphology of the immortalized sebocytes SZ95 according to Example 1 was epithelial and exhibited a polymorphous appearance with cells of different size, whereas numerous droplets could be observed in the cytoplasm (see FIGS. 1b and 1c).

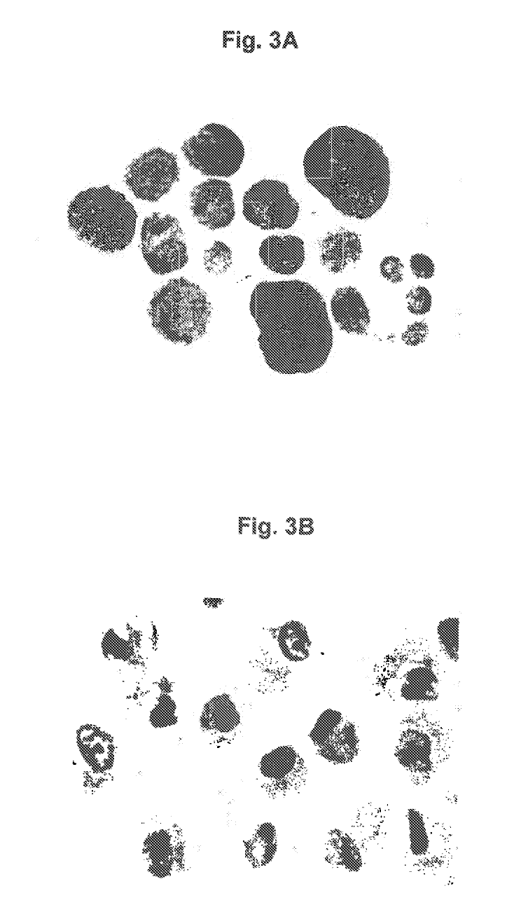

Immunocytochemical experiments of the immortalized sebocytes according to the present invention with respective antibodies resulted in a positive finding against the sebaceous gland antigene in contrast to normal epidermal keratinocytes which were not stained by the monoclonal antibody against the sebaceous gland antigene (see FIG. 3). FIG. 3 shows the immunocytochemical results on cytocentrifugation preparations (a) of the immortalized sebocytes according to the present invention as well as (b) normal human epidermal keratinocytes. The preparations were stained with an monoclonal antibody against the sebaceous gland antigene While the immortalized sebocytes of the present invention exhibited a positive cytoplasmatic staining, the normal human epidermal keratinocytes were not stained.

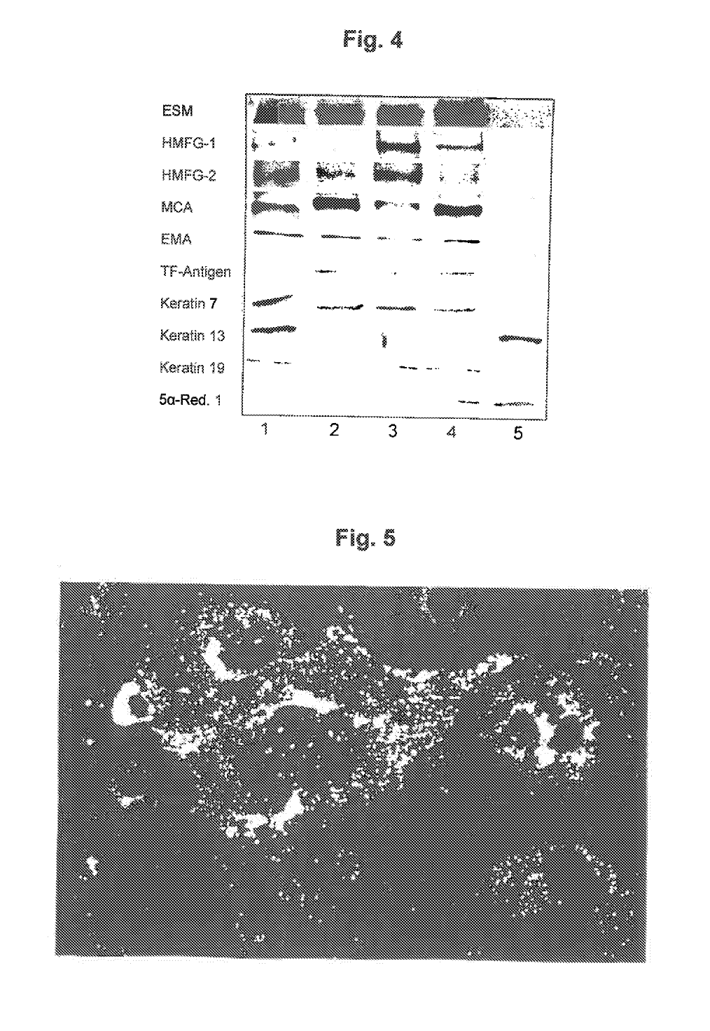

Moreover, the expression of the keratines 7, 13 and 19 as well as various proteins of the human polymorphous epithelial mucine group in the immortalized sebocytes was detected by Western blot analysis, whereas human keratinocytes expressed only keratine 13 (see FIG. 4). In Western blot analysis according to FIG. 4, extracted total protein of immortalized sebocytes according to the present invention (34^{th} sub-culture; lane 1), various cloned immortalized sebocytes according to the present invention (lanes 2, 3 and 4) as well as normal human epidermal keratinocytes (lane 5) were applied for identification of the expression of human epithelial sialomucin (ESM) (>400 kD), human milk fat globuline-1 (HMFG-1) (400 kD), human milk fat globuline-2 (HMFG-2) (80-400 kD), mucine-type carcinome-associated antigens (MCA) (350 kD), epithelial membrane antigene (EMA) (250-400 kD), Thomsen-Friedenreich antigene (TF antigens) (155 kD), Keratin 7 (54 kD), Keratin 13 (54 kD), Keratin 19 (40 kD) as well as 5α-reductase of type 1 (5α-Red.1) (21-27 kD). The immortalized cell line and its clones according to the present invention expressed all tested proteins, whereas keratinocytes only expressed Keratin 13 and 5.alpha.-reductase of type 1.

### Lipid Synthesis

The staining with Nile Red and the assessment by fluorescence microscopy showed the presence of lipids in the cell cytoplasm. In the immortalized sebocytes SZ95 of the present invention (see FIG. 5), stained by Nile Red fluorescent dye directed to neutral lipids resulted in individual or in grouped lipid droplets which were optionally divided in the cytoplasm of the sebocytes The immortalized sebocytes according to the present invention decreased their content from 510 fluorescent units per cell (median value) in serum-containing medium to 429 fluorescent units per cell (median value; i.e. −16%) in serum-free medium detected by fluorescence cytometric experiments of cells stained with Nile Red.

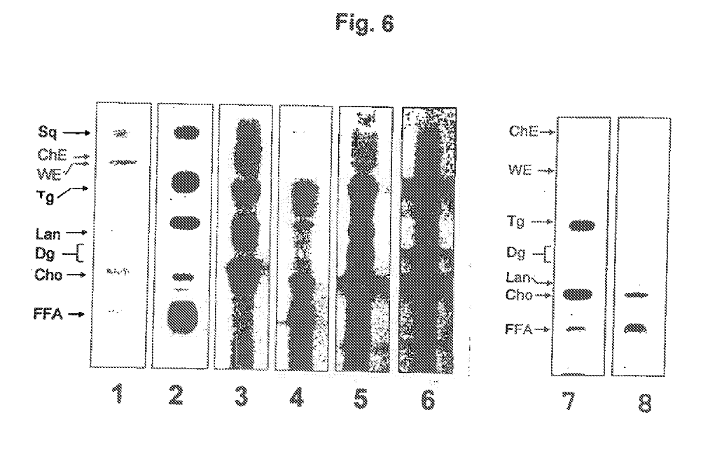

The immortalized sebocytes according to the present invention of Example 1 synthesized various fractions of neutral lipids including the typical sebaceous gland lipids squalene and wax ester as well as triglycerides, cholesterol, cholesterol ester, diglycerides, lanosterol and free fatty acids. This was observed throughout 25 to 40 sub-cultures. The result in shown in FIG. 6, where HPTLC-fractionized lipids were detected after pulse recording of radioactively labeled sodium acetate by means of radiometric image evaluation with two selected immortalized and cloned sebocyte cultures (see lanes 3 and 4 as well as 5 and 6, respectively). As shown by lanes 3, 4 and 5, the cells synthesized multiple fractions of neutral lipids including squalene (Sq), wax ester (WE) as well as triglycerides (Tg), cholesterol (Cho), cholesterol ester (ChE), diglycerides (Dg), lanosterol (Lan) as well as free fatty acids (FFA). All neutral lipids were also found but in a lesser extend in the extracellular supernatant (see lane 6). For comparison in lane 1, lipid standards, in lane 2 human sebum, and in lane 4, free fatty acids extracted from cells were applied. As further comparison, lanes 7 and 8 showed the results of keratinocytes, whereas lane 7 showed the presence mainly of cholesterol and triglycerides in the cells, while from the supernatant (lane 8), mostly cholesterine was found.

### Proliferation Studies

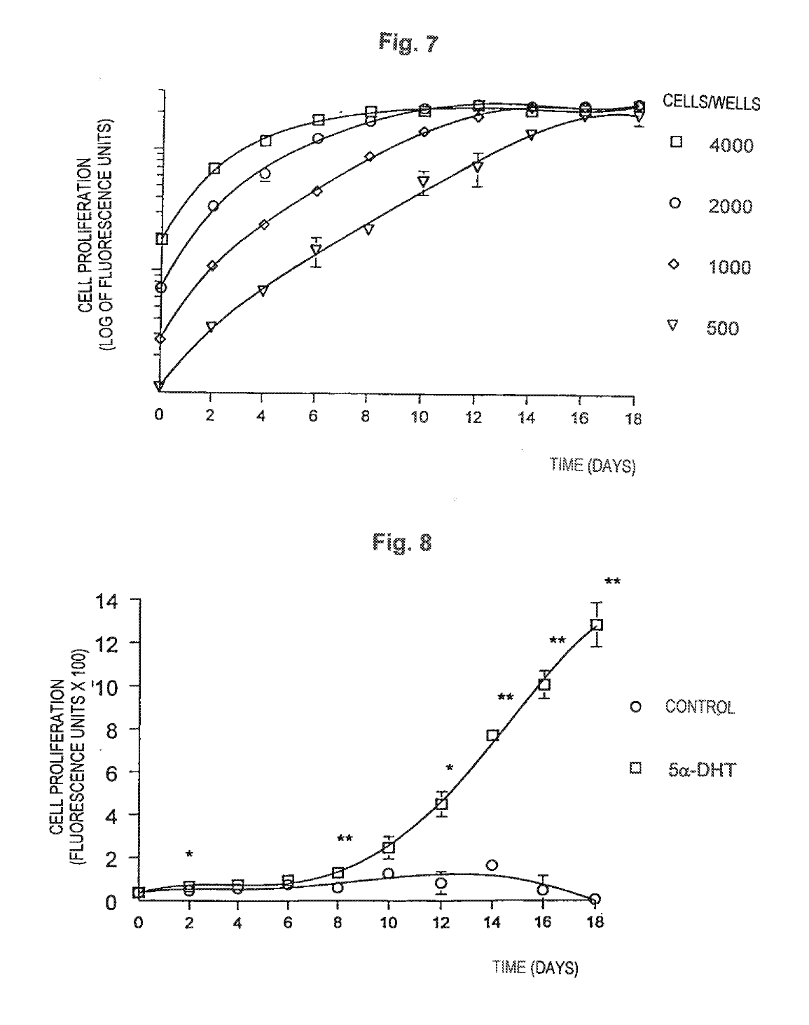

A logarithmic proliferation pattern of the immortalized sebocytes according to the present invention was detected under normal culture conditions with population doubling times of 52.4+−1.6 independent from the original culture cell densities. To this extent, FIG. 7 shows the proliferation of an immortalized cloned sebocyte cell line (SZ95) over 18 days in sebocyte medium.

The proliferation of the immortalized sebocytes was reduced after addition of serum-free medium but was retrieved after addition of 5α-DHT. This is shown in FIG. 8 for an exemplary sebocyte clone where the proliferation of the cells (seeding of 2.000/well) was observed over 18 days in serum-free medium (control) as well as in serum-free medium which was supplemented with 10⁻⁶ M 5α-DHT. After the 8^{th} day, 5α-DHT increased the proliferation of the cells significantly which was shown by the determined population doubling time of 136 hours (control) and 53.7 hours (5α-DHT-treated cells (*, p<0.05; **, p<0.01).

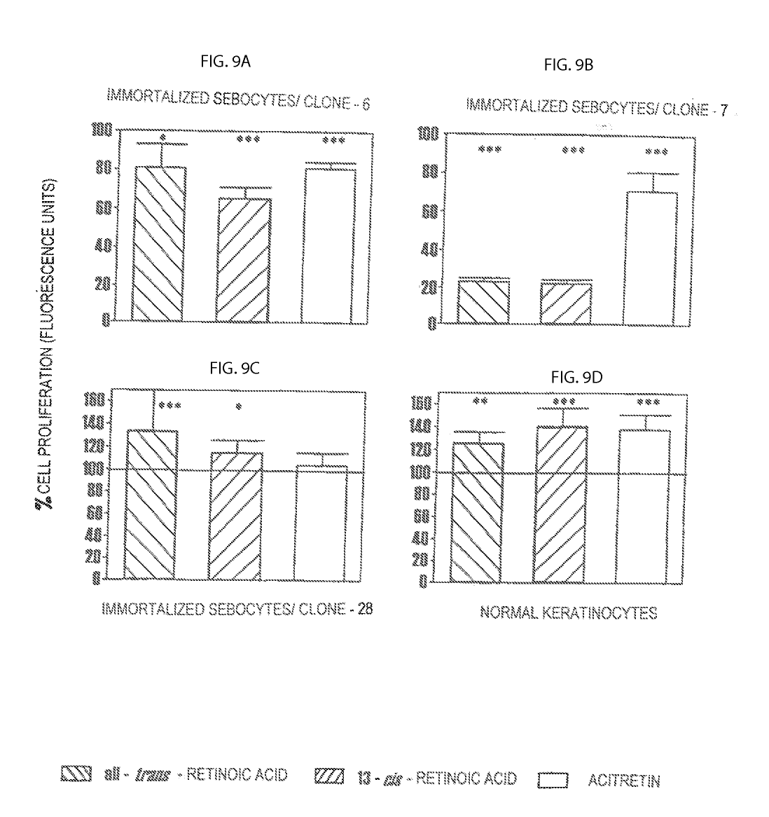

The influence of retinoids on the immortalized cells showed a differentiated response of the proliferation behavior. While some clones showed inhibition of proliferation by retinoids (typically distinctively pronounced in the order of 13-cis-retinoic acid>all-trans-retinoic acid>>Acitretin), other clones were stimulated in proliferation (for example by all-trans-retinoic acid and 13-cis-retinoic acid) corresponding to the proliferation response of normal human epidermal keratincoytes. This is shown in FIG. 9 (*, p<0.05; **, p<0.01; ***, p<0.001).

1. An immortalized sebocyte derived from a human.

2. The sebocyte according to claim 1, wherein it is derived from a human sebaceous gland cell.

3. The sebocyte according to claim 2, wherein the sebaceous gland cell is a facial sebaceous gland cell.

4. The sebocyte according to claim 1, wherein it is present in the form of a cell line.

5. The sebocyte according to claim 1, wherein it is immortalized by transfection of DNA.

6. The sebocyte according to claim 1, wherein it expresses a SV-40 large T antigen.

7. The sebocyte according to claim 1, wherein it exhibits features of a normal, non-transfected and differentiating sebocyte.

8. The sebocyte according to claim 1, wherein its proliferation is modifiable by an antigen and/or a retinoid.

9. The sebocyte according to claim 1, wherein it is cloned.

10. Human sebocyte cell line DSM ACC2383.

11. Use of the sebocyte according to claim 1, for diagnostic, therapeutic or cosmetic preparations.

12. Use of the sebocyte according to claim 1, for the examination of the physiology or the pathophysiology of human or animal sebaceous gland.

13. Use of the sebocyte according to claim 1 for the examination of the origin of acne and/or seborrhoe and/or other diseases.

14. Use of the sebocyte according to claim 13, wherein the other diseases to be examined are dermal diseases in which the sebaceous gland function is involved or may be involved.

15. Use of the sebocyte according to claim 1, for the testing of anti-acne and/or anti-seborrhoe compounds or agents.

16. Use of the sebocyte according to claim 1, for the testing of compounds or agents against diseases.

17. Use of the sebocyte according to claim 16, wherein the diseases are dermal diseases in which the sebaceous gland function is involved or may be involved.

18. Use of the sebocyte according to claim 1, for the development of simple or complex cell culture systems.

19. Use of the sebocyte according to claim 1, for the formation of, or for the use in, three-dimensional cell aggregations or constructions of organ-type structures.

20. Use of the sebocyte according to claim 1, for the preparation of products derived from said cells.

21. Use according to claim 20, wherein the cell products are lipids, plasmids, vectors, proteins which are expressed by said cells and/or DNA or RNA sequences of said proteins.

22. Use of the products obtained according to claim 20 for the modification of other cells or the modification of organisms.

23. Use of the human sebocyte cell line according to claim 10, for diagnostic, therapeutic or cosmetic preparations.

24. Use of the human sebocyte cell line according to claim 10, for the examination of the physiology or the pathophysiology of human or animal sebaceous gland.

25. Use of the human sebocyte cell line according to claim 10, for the examination of the origin of acne and/or seborrhoe and/or other diseases.

26. Use of the human sebocyte cell line according to claim 10, for the testing of anti-acne and/or anti-seborrhoe compounds or agents.

27. Use of the human sebocyte cell line according to claim 10, for the testing of compounds or agents against diseases.

28. Use of the human sebocyte cell line according to claim 10, for the development of simple or complex cell culture systems.

29. Use of the human sebocyte cell line according to claim 10, for the formation of, or for the use in, three-dimensional cell aggregations or constructions of organ-type structures.

30. Use of the human sebocyte cell line according to claim 10, for the preparation of products derived from said cells.

31. Use of the products obtained according to claim 21 for the modification of other cells or the modification of organisms.
